(19) United States
(12) Patent Application Publication
(10) Pub. No.: US 2026/0130922 A1
(43) Pub. Date: May 14, 2026

(54) METHODS OF TREATING CANCER USING AN ANTI-CTLA4 ANTIBODY AND AN ENPP1 INHIBITOR

(71) Applicant: AGENUS INC., Lexington, MA (US)

(72) Inventors: Jaymin Patel, Lexington, MA (US); Steven O'Day, Lexington, MA (US); Dhan Sidhartha Chand, Lexington, MA (US)

(21) Appl. No.: 19/355,137

(22) Filed: Oct. 10, 2025

Related U.S. Application Data
(63) Continuation of application No. PCT/US2024/024264, filed on Apr. 12, 2024.
(60) Provisional application No. 63/495,666, filed on Apr. 12, 2023.
(60) Provisional application No. 63/595,619, filed on Nov. 2, 2023.

(51) Int. Cl. A61K 31/551; A61K 39/00; A61P 35/00; C07K 16/28

(52) U.S. Cl. CPC A61K 31/551; A61P 35/00; C07K 16/2818; A61K 2039/507; A61K 2039/545; C07K 2317/565; C07K 2317/72

(57) ABSTRACT

Provided are methods for treating cancer with an antibody that specifically binds to human Cytotoxic T-Lymphocyte Antigen 4 (CTLA-4) and an ectonucleotide pyrophosphatase/phosphodiesterase family member 1 (ENPP1) inhibitor.

### RELATED APPLICATIONS

This application is a continuation of International Application Serial No. PCT/US2024/024264, filed Apr. 12, 2024, which claims priority to U.S. Provisional Patent Application Ser. No. 63/495,666, filed Apr. 12, 2023, and to U.S. Provisional Patent Application Ser. No. 63/595,619, filed Nov. 2, 2023, the entire disclosures of which are hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Oct. 10, 2025, is named “222233_seqlist.xml” and is 10,403 bytes in size).

### BACKGROUND

Checkpoint inhibitors (CIs), such as anti-CTLA-4 antibodies, increase immune response to tumor cells by activating the adaptive immune response. CIs have shown promise in the clinic, but not all cancer types respond to CI therapy. However, activation of the innate immune response may help sensitize multiple cancer types to adaptive immune therapies, such as CIs.

The cyclic GMP-AMP synthase (cGAS)-stimulator of interferon genes (STING) pathway, which is activated in response to cytosolic DNA, has emerged as a key mechanism to activate innate immunity, primarily through type I interferon (IFN) signaling. Several direct STING agonists have been developed but have not performed well in the clinic. This is likely because of the widespread expression of STING in normal tissues, where the hyperactivation of STING can lead to a systemic cytokine storm. ENPP1 is the only known direct negative regulator of the STING pathway that hydrolyzes 2′3′ cGAMP, the direct ligand of STING. High levels of ENPP1 expression in multiple cancer types might promote resistance to CIs. Therefore, ENPP1 inhibitors might present an alternative approach to enhance the effect of CIs by activating the STING pathway and thus, activating the innate immune response. The highest levels of 2′3′ cGAMP are found in tumors and recent evidence suggests that 2′3′ cGAMP acts locally, as a paracrine immune transmitter. Therefore, inhibition of ENPP1 may produce superior outcomes by activating STING in the tumor microenvironment, without hyperactivating the STING pathway in normal tissues.

Thus, the combination of an ENPP1 inhibitor and a checkpoint inhibitor presents a promising approach for treating cancer.

### SUMMARY

The instant disclosure is directed to methods for treating cancer with an antibody that specifically binds to human Cytotoxic T-Lymphocyte Antigen 4 (CTLA-4) in combination with an ectonucleotide pyrophosphatase/phosphodiesterase-1 (ENPP1) inhibitor and therapeutic combinations comprising an antibody that specifically binds to human CTLA-4 and an ENPP1 inhibitor. Also provided herein are particular methods for administering an antibody that specifically binds to human CTLA-4 and an ENPP1 inhibitor that results in a reduction of tumor burden in a subject.

In an aspect, provided herein is a method of treating cancer in a subject in need thereof, the method comprising administering to the subject a therapeutically effective amount of: (a) an antibody that specifically binds to human Cytotoxic T-Lymphocyte Antigen 4 (CTLA-4); and (b) an ectonucleotide pyrophosphatase/phosphodiesterase family member 1 (ENPP1) inhibitor.

In an aspect, provided herein is a method of enhancing the activation of T cells in a subject who has cancer, the method comprising administering to the subject a therapeutically effective amount of: (a) an antibody that specifically binds to human Cytotoxic T-Lymphocyte Antigen 4 (CTLA-4); and (b) an ectonucleotide pyrophosphatase/phosphodiesterase family member 1 (ENPP1) inhibitor.

In an embodiment, the antibody comprises a human IgG₁ heavy chain constant region that is a variant of a wild-type human IgG heavy chain constant region, wherein the variant binds to FcγRIIIA with a higher affinity than the wild-type human IgG₁ heavy chain constant region binds to FcγRIIIA.

In an embodiment, the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a human IgG₁ heavy chain constant region comprising S239D/A330L/I332E mutations, numbered according to the EU numbering system. In an embodiment, the antibody comprises a heavy chain comprising the amino acid sequence set forth in SEQ ID NO: 9 and a light chain comprising the amino acid sequence set forth in SEQ ID NO: 10.

In an embodiment, the antibody that specifically binds to human CTLA-4 is botensilimab.

In an embodiment, the ENPP1 inhibitor is:

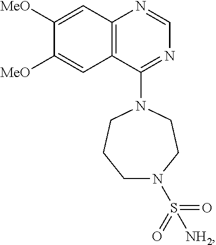 [SMILES: COc1cc2ncnc(N3CCCN(S(N)(=O)=O)CC3)c2cc1OC]

or a pharmaceutically acceptable salt thereof.

In an embodiment, the antibody is administered to the subject at a dose of 25 mg to 250 mg. In an embodiment, the antibody is administered to the subject at a dose of 25 mg, 50 mg, 75 mg, 100 mg, 150 mg, 200 mg, or 250 mg.

In an embodiment, the antibody is administered to the subject intravenously, subcutaneously, or intratumorally.

In an embodiment, the antibody is administered to the subject once weekly. In an embodiment, the antibody is administered to the subject once every 2 weeks. In an embodiment, the antibody is administered to the subject once every 3 weeks. In an embodiment, the antibody is administered to the subject once every 4 weeks. In an embodiment, the antibody is administered to the subject once every 5 weeks. In an embodiment, the antibody is administered to the subject once every 6 weeks.

In an embodiment, the ENPP1 inhibitor is administered to the subject at a dose of 5 mg/day to 1000 mg/day. In an embodiment, the ENPP1 inhibitor is administered to the subject at a dose of 10 mg/day to 50 mg/day. In an embodiment, the ENPP1 inhibitor is administered to the subject at a dose of 10 mg/day, 20 mg/day, 30 mg/day, 40 mg/day, or 50 mg/day.

In an embodiment, the cancer is bladder cancer, breast cancer, cervical cancer, colorectal cancer, colon adenocarcinoma, head and neck cancer, leukemia, lymphoma, tenosynovial giant cell tumor, gastric cancer, gastroesophageal cancer, glioblastoma, sarcoma, pancreatic cancer, melanoma, mesothelioma, renal cell adenocarcinoma, hepatocellular carcinoma, stomach adenocarcinoma, kidney renal clear cell carcinoma, esophageal carcinoma, ovarian cancer, small cell lung cancer, non-small cell lung cancer, or lung adenocarcinoma.

In an embodiment, the cancer is relapsed and/or refractory. In an embodiment, the cancer is metastatic. In an embodiment, the cancer is microsatellite stable colorectal cancer. In an embodiment, the cancer is renal cell carcinoma. In an embodiment, the cancer is hepatocellular carcinoma. In an embodiment, the cancer is pancreatic cancer.

In an embodiment, the subject has received at least one prior immunotherapy or chemotherapy. In an embodiment, the subject has not received any prior chemotherapy, radiotherapy, or immunotherapy.

In an embodiment, the cancer is refractory to a standard of care treatment. In an embodiment, the standard of care treatment is chemotherapy, immunotherapy, or radiation.

In an embodiment, the method further comprises administering an additional therapeutic agent to the subject. In an embodiment, the additional therapeutic agent is an antibody that specifically binds to human PD-1. In an embodiment, the additional therapeutic agent is nivolumab, pembrolizumab, dostarlimab, or balstilimab.

In an embodiment, the method reduces tumor size in the subject. In an embodiment, the method increases T-cell activation in the subject.

In an aspect, disclosed herein is an antibody that specifically binds to human CTLA-4 and an ENPP1 inhibitor for use in the treatment of cancer, wherein the treatment is performed according to any one of the methods described herein.

In an aspect, disclosed herein is an antibody that specifically binds to human CTLA-4 and an ENPP1 inhibitor for use in the manufacture of a medicament for the treatment of cancer, wherein the treatment is performed according to any one of the methods described herein.

In an aspect, disclosed herein is a use of an antibody that specifically binds to human CTLA-4 and an ENPP1 inhibitor for the treatment of cancer, wherein the treatment is performed according to any one of the methods described herein.

In an aspect, disclosed herein is an antibody that specifically binds to human CTLA-4 and an ENPP1 inhibitor for use in enhancing the activation of T cells, wherein the treatment is performed according to any one of the methods described herein.

In an aspect, disclosed herein is an antibody that specifically binds to human CTLA-4 and an ENPP1 inhibitor for use in the manufacture of a medicament for enhancing the activation of T cells, wherein the treatment is performed according to any one of the methods described herein.

In an aspect, disclosed herein is a use of an antibody that specifically binds to human CTLA-4 and an ENPP1 inhibitor for enhancing the activation of T cells, wherein the treatment is performed according to any one of the methods described herein.

In an aspect, disclosed herein is a therapeutic combination comprising: (a) an antibody that specifically binds to human Cytotoxic T-Lymphocyte Antigen 4 (CTLA-4); and (b) an ectonucleotide pyrophosphatase/phosphodiesterase family member 1 (ENPP1) inhibitor.

In an embodiment, the antibody comprises a human IgG₁ heavy chain constant region that is a variant of a wild-type human IgG heavy chain constant region, wherein the variant binds to FcγRIIIA with a higher affinity than the wild-type human IgG₁ heavy chain constant region binds to FcγRIIIA.

In an embodiment, the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a human IgG1 heavy chain constant region comprising S239D/A330L/I332E mutations, numbered according to the EU numbering system. In an embodiment, the antibody comprises a heavy chain comprising the amino acid sequence set forth in SEQ ID NO: 9 and a light chain comprising the amino acid sequence set forth in SEQ ID NO: 10.

In an embodiment, the antibody is botensilimab.

In an embodiment, the ENPP1 inhibitor is:

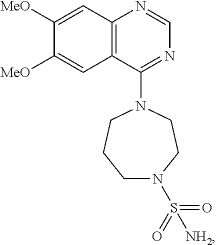 [SMILES: COc1cc2ncnc(N3CCCN(S(N)(=O)=O)CC3)c2cc1OC]

or a pharmaceutically acceptable salt thereof.

### BRIEF DESCRIPTION OF THE DRAWINGS

FIG. 1A is a graph showing the tumor growth curves of mice treated with either isotype/vehicle control, botensilimab^{ms}, compound 1, or a combination of botensilimab^{ms} and compound 1. FIG. 1B is a graph showing the mouse body weights during the treatment. FIGS. 1C-1F are graphs showing the tumor growth curves of individual mice treated with either isotype/vehicle control (FIG. 1C), botensilimab^{ms} (FIG. 1D), compound 1 (FIG. 1E), or a combination of botensilimab^{ms} and compound 1 (FIG. 1F).

FIGS. 2A and 2B are graphs showing the percent of CD3+ cells (FIG. 2A) and CD8+ cells (FIG. 2B) in tumors from mice treated with isotype/vehicle control, botensilimab^{ms}, compound 1, or a combination of botensilimab^{ms} and compound 1. Data presented is mean±SEM; *p<0.05, **p<0.01, ****p<0.0001; one-way ANOVA, Tukey's multiple comparisons test.

FIGS. 3A-3E are graphs showing gene expression of IFNβ (FIG. 3A), IFNγ (FIG. 3B), CXCL10 (FIG. 3C), ISG56 (FIG. 3D), and Granzyme B (FIG. 3E) in tumors from mice treated with isotype/vehicle control, botensilimab^{ms}, compound 1, or a combination of botensilimab^{ms} and compound 1. Data presented is mean±SEM; *p<0.05, **p<0.01, ****p<0.0001; one-way ANOVA, Tukey's multiple comparisons test; ns means not significant.

FIG. 4 is a graph showing tumor growth curves for female BALB/cJ mice implanted subcutaneously with CT-26 cells, and treated with either isotype/vehicle control, botensilimab^{ms}, compound 1, or a combination of botensilimab^{ms} and compound 1.

FIGS. 5A and 5B are graphs showing gene (mRNA) expression of the immune pathway marker IFNβ, in tumors from mice treated with isotype/vehicle control, botensilimab^{ms}, compound 1, or a combination of botensilimab^{ms} and compound 1, at day 4 (FIG. 5A) and day 8 (FIG. 5B) after initiation of dosing. Data presented is mean±SEM; one-way ANOVA, Tukey's multiple comparisons test; ns means not significant.

FIGS. 6A and 6B are graphs showing gene (mRNA) expression of the immune pathway marker IFNγ, in tumors from mice treated with isotype/vehicle control, botensilimab^{ms}, compound 1, or a combination of botensilimab^{ms} and compound 1, at day 4 (FIG. 6A) and day 8 (FIG. 6B) after initiation of dosing. Data presented is mean±SEM; *p<0.05, ****p<0.0001; one-way ANOVA, Tukey's multiple comparisons test; ns means not significant.

FIGS. 7A and 7B are graphs showing gene (mRNA) expression of the immune pathway marker ISG15, in tumors from mice treated with isotype/vehicle control, botensilimab^{ms}, compound 1, or a combination of botensilimab^{ms} and compound 1, at day 4 (FIG. 7A) and day 8 (FIG. 7B) after initiation of dosing. Data presented is mean±SEM; **p<0.01; one-way ANOVA, Tukey's multiple comparisons test; ns means not significant.

FIGS. 8A and 8B are graphs showing gene (mRNA) expression of the immune pathway marker ISG56, in tumors from mice treated with isotype/vehicle control, botonsilimab^{ms}, compound 1, or a combination of botensilimab^{ms} and compound 1, at day 4 (FIG. 8A) and day 8 (FIG. 8B) after initiation of dosing. Data presented is mean±SEM; *p<0.05, **p<0.01; one-way ANOVA, Tukey's multiple comparisons test; ns means not significant.

FIGS. 9A and 9B are graphs showing gene (mRNA) expression of the immune pathway marker CXCL10, in tumors from mice treated with isotype/vehicle control, botensilimab^{ms}, compound 1, or a combination of botensilimab^{ms} and compound 1, at day 4 (FIG. 9A) and day 8 (FIG. 9B) after initiation of dosing. Data presented is mean±SEM; **p<0.01, ****p<0.0001; one-way ANOVA, Tukey's multiple comparisons test; ns means not significant.

FIGS. 10A and 10B are graphs showing gene (mRNA) expression of the immune pathway marker Granzyme B, in tumors from mice treated with isotype/vehicle control, botensilimab^{ms}, compound 1, or a combination of botensilimab^{ms} and compound 1, at day 4 (FIG. 10A) and day 8 (FIG. 10B) after initiation of dosing. Data presented is mean±SEM; **p<0.01; one-way ANOVA, Tukey's multiple comparisons test; ns means not significant.

FIGS. 11A-11H are graphs showing the tumor growth curves of individual mice treated with either isotype/vehicle control (FIG. 11A), botonsilimab^{ms} (“Bot^{MS}”) (FIG. 11B), an anti-PD-1 antibody (“α-PD1”) (FIG. 11C), Compound 1 (FIG. 11D), a combination of botensilimab^{ms} and Compound 1 (“Compound 1+Bot^{MS}”) (FIG. 11E), a combination of Compound 1 and anti-PD-1 antibody (“Compound 1+α-PD1”) (FIG. 11F), a combination of botensilimab^{ms} and anti-PD-1 antibody (“Bot^{MS}+α-PD1”) (FIG. 11G), or a combination of Compound 1, botensilimab^{ms} and anti-PD-1 antibody (“Compound 1+Bot^{MS}+α-PD1”) (FIG. 11H), monitored over 40 days. CR indicates the number of complete responses observed out of all treated mice.

### DETAILED DESCRIPTION

The instant disclosure is directed to methods for treating cancer with an antibody that specifically binds to human Cytotoxic T-Lymphocyte Antigen 4 (CTLA-4) in combination with an ectonucleotide pyrophosphatase/phosphodiesterase-1 (ENPP1) inhibitor and therapeutic combinations comprising an antibody that specifically binds to human CTLA-4 and an ENPP1 inhibitor. Also provided herein are particular methods for administering an antibody that specifically binds to human CTLA-4 and an ENPP1 inhibitor that results in a reduction of tumor burden in a subject.

### Definitions

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F (ab′) 2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgAQ1, or IgA2), or any subclass (e.g., IgG2a or lgG2b) of immunoglobulin molecule. In certain embodiments, antibodies described herein are IgG antibodies, or a class (e.g., human IgG1 or IgG4) or subclass thereof. In an embodiment, the antibody is a humanized monoclonal antibody. In an embodiment, the antibody is a human monoclonal antibody.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of Proteins of Immunological Interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other (see Table 1 below). In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of Proteins of Immunological Interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human CTLA-4). CDRH1, CDRH2, and CDRH3 denote the heavy chain CDRs, and CDRL1, CDRL2, and CDRL3 denote the light chain CDRs.

TABLE 1
CDR definitions

| | CDR Definitions | | |
|---|---|---|---|
| | Kabat | Chothia | MacCallum |
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (α), delta (ε), epsilon (γ), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In an embodiment, molecules that specifically bind to an antigen bind to the antigen with a KA that is at least 2 logs (e.g., factors of 10), 2.5 logs, 3 logs, 4 logs or greater than the KA when the molecules bind non-specifically to another antigen.

As used herein, the term “EU numbering system” refers to the EU numbering convention for the constant regions of an antibody, as described in Edelman G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “subject” includes any human or non-human animal. In an embodiment, the subject is a human.

As used herein, the term “effective amount” in the context of the administration of a therapy to a subject refers to the amount of a therapy that achieves a desired prophylactic or therapeutic effect.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “standard of care” refers to the most common treatments prescribed for a particular type of cancer.

As used herein, the term “tumor burden” refers to the number of cancer cells, the size of a tumor, or the amount of cancer in the body of the subject.

As used herein, the term “about” when referring to a measurable value, such as a dosage, encompasses variations of +20%, +15%, +10%, +5%, +1%, or +0.1% of a given value or range, as are appropriate to perform the methods disclosed herein.

### Anti-CTLA-4 Antibodies

Antibodies that specifically bind to human CTLA-4 (i.e., anti-CTLA-4 antibodies) that are useful in the methods, uses, and therapeutic combinations described herein include but are not limited to those listed below.

In an embodiment, the antibody comprises a human IgG₁ heavy chain constant region that is a variant of a wild-type human IgG heavy chain constant region, wherein the variant binds to FcγRIIIA with a higher affinity than the wild-type human IgG₁ heavy chain constant region binds to FcγRIIIA.

In an embodiment, the antibody comprises: a heavy chain variable region (VH) comprising the CDRH1, CDRH2, and CDRH3 amino acid sequences of the VH amino acid sequence set forth in SEQ ID NO: 7. In an embodiment, the antibody comprises a heavy chain variable region (VH) comprising the CDRH1, CDRH2, and CDRH3 amino acid sequences of the VH amino acid sequence set forth in SEQ ID NO: 7 and a light chain variable region (VL) comprising the CDRL1, CDRL2, and CDRL3 amino acid sequences of the VL amino acid sequence set forth in SEQ ID NO: 8.

In an embodiment, the antibody comprises the CDRH1, CDRH2, and CDRH3 amino acid sequences set forth in SEQ ID NO: 1, 2, and 3, respectively. In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7. In an embodiment, the antibody comprises: a VH comprising the amino acid sequence set forth in SEQ ID NO: 7; and a VL comprising an amino acid sequence which is at least 90%, 95%, 96%, 97%, 98%, 99%, or 100% identical to the amino acid sequence set forth in SEQ ID NO: 8.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a heavy chain constant region selected from the group consisting of human IgG₁, IgG₂, IgG₃, IgG₄, IgA₁, and IgA₂. In an embodiment, the heavy chain constant region is IgG₁. In an embodiment, the heavy chain constant region is IgG₂. In an embodiment, the antibody comprises a human kappa light chain constant region or a human lambda light chain constant region.

In an embodiment, the antibody comprises an IgG₁ heavy chain constant region. In an embodiment, the amino acid sequence of the IgG₁ heavy chain constant region comprises S239D/I332E mutations, numbered according to the EU numbering system. In an embodiment, the amino acid sequence of the IgG₁ heavy chain constant region comprises S239D/A330L/I332E mutations, numbered according to the EU numbering system. In an embodiment, the amino acid sequence of the IgG₁ heavy chain constant region comprises L235V/F243L/R292P/Y300L/P396L mutations, numbered according to the EU numbering system. In an embodiment, the IgG₁ heavy chain constant region is afucosylated IgG₁.

In an embodiment, the antibody comprises a heavy chain comprising the amino acid sequence set forth in SEQ ID NO: 9. In an embodiment, the antibody comprises a heavy chain comprising the amino acid sequence set forth in SEQ ID NO: 9 and a light chain comprising an amino acid sequence which is at least 90%, 95%, 96%, 97%, 98%, 99%, or 100% identical to the amino acid sequence set forth in SEQ ID NO: 10.

In an embodiment, the antibody comprises a heavy chain comprising the amino acid sequence set forth in SEQ ID NO: 9 and a light chain comprising the amino acid sequence set forth in SEQ ID NO: 10. In an embodiment, the amino acid sequence of the heavy chain consists of the amino acid sequence set forth in SEQ ID NO: 9 and the amino acid sequence of the light chain consists of the amino acid sequence set forth in SEQ ID NO: 10.

In an embodiment the antibody is botensilimab (a.k.a. AGEN1181), the amino acid sequences of which are provided in Table 2 below.

TABLE 2
Amino acid sequences of botensilimab

| | SEQ ID | |
|---|---|---|
| Description | NO: | Amino acid sequence |
| CDRH1 | 1 | SYSMN |
| CDRH2 | 2 | SISSSSSYIYYAESVKG |
| CDRH3 | 3 | VGLFGPFDI |
| CDRL1 | 4 | RASQSVSRYLG |
| CDRL2 | 5 | GASTRAT |
| CDRL3 | 6 | QQYGSSPWT |
| VH | 7 | EVQLVESGGGLVKPGGSLRLSCAASGFTFSSYSMNWVRQ |
| | | APGKGLEWVSSISSSSSYIYYAESVKGRFTISRDNAKNSLY |
| | | LQMNSLRAEDTAVYYCARVGLFGPFDIWGQGTLVTVSS |
| VL | 8 | EIVLTQSPGTLSLSPGERATLSCRASQSVSRYLGWYQQKP |
| | | GQAPRLLIYGASTRATGIPDRFSGSGSGTDFTLTITRLEPE |
| | | DFAVYYCQQYGSSPWTFGQGTKVEIK |
| Heavy chain | 9 | EVQLVESGGGLVKPGGSLRLSCAASGFTFSSYSMNWVRQ |
| | | APGKGLEWVSSISSSSSYIYYAESVKGRFTISRDNAKNSLY |
| | | LQMNSLRAEDTAVYYCARVGLFGPFDIWGQGTLVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKRVEPKSCDKTHTCPPCPAPELLG |
| | | GPDVFLFPPKPKDTLMISRTPEVTCVVVDVSHEDPEVKFN |
| | | WYVDGVEVHNAKTKPREEQYNSTYRVVSVLTVLHQDW |
| | | LNGKEYKCKVSNKALPLPEEKTISKAKGQPREPQVYTLPP |
| | | SREEMTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPG |
| Light chain | 10 | EIVLTQSPGTLSLSPGERATLSCRASQSVSRYLGWYQQKP |
| | | GQAPRLLIYGASTRATGIPDRFSGSGSGTDFTLTITRLEPE |
| | | DFAVYYCQQYGSSPWTFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |

In an embodiment, the antibody is antagonistic to human CTLA-4. In an embodiment, the antibody deactivates, reduces, or inhibits an activity of human CTLA-4. In an embodiment, the antibody inhibits binding of human CTLA-4 to human CD80 or human CD86. In an embodiment, the antibody induces IL-2 production by peripheral blood mononuclear cells (PBMCs) stimulated with staphylococcal enterotoxin A (SEA).

### PD-1 Inhibitors

Human PD-1 inhibitors that are useful in the methods, uses, and therapeutic combinations described herein include but are not limited to those listed below.

In an embodiment, the human PD-1 inhibitor is an antibody that specifically binds to human PD-1. In an embodiment, the antibody that specifically binds to human PD-1 is balstilimab.

In an embodiment, the human PD-1 inhibitor is an antibody that specifically binds to human PD-L1.

In an embodiment, the antibody that specifically binds to human PD-1 or human PD-L1 is adebrelimab, atezolizumab, avelumab, camrelizumab, cemiplimab, cosibelimab, dostarlimab, durvalumab, enlonstobart, envafolimab, nivolumab, pembrolizumab, penpulimab, pidilizumab, prolgolimab, pucotenlimab, retifanlimab, serplulimab, sintilimab, socazolimab, sugemalimab, tagitanlimab, tislelizumab, toripalimab, and zimberelimab.

Further non-limiting examples of anti-PD-1 antibodies that may be used in treatment methods described herein are disclosed in the following patents and patent applications, which are incorporated herein by reference in their entireties for all purposes: U.S. Pat. Nos. 6,808,710; 7,332,582; 7,488,802; 8,008,449; 8,114,845; 8,168,757; 8,354,509; 8,686,119; 8,735,553; 8,747,847; 8,779,105; 8,927,697; 8,993,731; 9,102,727; 9,205,148; U.S. Publication No. US 2013/0202623 A1; U.S. Publication No. US 2013/0291136 A1; U.S. Publication No. US 2014/0044738 A1; U.S. Publication No. US 2014/0356363 A1; U.S. Publication No. US 2016/0075783 A1; and PCT Publication No. WO 2013/033091 A1; PCT Publication No. WO 2015/036394 A1; PCT Publication No. WO 2014/179664 A2; PCT Publication No. WO 2014/209804 A1; PCT Publication No. WO 2014/206107 A1; PCT Publication No. WO 2015/058573 A1; PCT Publication No. WO 2015/085847 A1; PCT Publication No. WO 2015/200119 A1; PCT Publication No. WO 2016/015685 A1; and PCT Publication No. WO 2016/020856 A1.

Further examples of anti-PD-L1 antibodies that may be used in treatment methods described herein are disclosed in the following patents and patent applications, which are incorporated herein by reference in their entireties for all purposes: U.S. Pat. Nos. 7,943,743; 8,168,179; 8,217,149; 8,552,154; 8,779,108; 8,981,063; 9,175,082; U.S. Publication No. US 2010/0203056 A1; U.S. Publication No. US 2003/0232323 A1; U.S. Publication No. US 2013/0323249 A1; U.S. Publication No. US 2014/0341917 A1; U.S. Publication No. US 2014/0044738 A1; U.S. Publication No. US 2015/0203580 A1; U.S. Publication No. US 2015/0225483 A1; U.S. Publication No. US 2015/0346208 A1; U.S. Publication No. US 2015/0355184 A1; and PCT Publication No. WO 2014/100079 A1; PCT Publication No. WO 2014/022758 A1; PCT Publication No. WO 2014/055897 A2; PCT Publication No. WO 2015/061668 A1; PCT Publication No. WO 2015/109124 A1; PCT Publication No. WO 2015/195163 A1; PCT Publication No. WO 2016/000619 A1; and PCT Publication No. WO 2016/030350 A1.

In an embodiment, the human PD-1 inhibitor is pidilizumab.

### ENPP1 Inhibitors

Ectonucleotide pyrophosphatase/phosphodiesterase family member 1 (ENPP1) inhibitors that are useful in the methods, uses, and therapeutic combinations described herein include but are not limited to those listed below.

In an embodiment, the ENPP1 inhibitor deactivates, reduces, or inhibits an activity of human ENPP1. In an embodiment, the ENPP1 inhibitor inhibits the hydrolase activity of ENPP1. In an embodiment, the ENPP1 inhibitor activates the STING pathway.

In an embodiment, the ENPP1 inhibitor is compound 1:

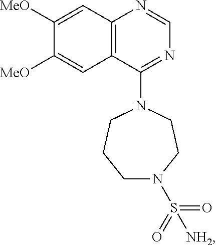 [SMILES: COc1cc2ncnc(N3CCCN(S(N)(=O)=O)CC3)c2cc1OC]

or a pharmaceutically acceptable salt thereof.

### Methods of Treatment

In an aspect, provided herein is a method of treating cancer in a subject in need thereof, the method comprising administering to the subject a therapeutically effective amount of: (a) an antibody that specifically binds to human Cytotoxic T-Lymphocyte Antigen 4 (CTLA-4); and (b) an ectonucleotide pyrophosphatase/phosphodiesterase family member 1 (ENPP1) inhibitor.

In an aspect, provided herein is a method of enhancing the activation of T cells in a subject who has cancer, the method comprising administering to the subject a therapeutically effective amount of: (a) an antibody that specifically binds to human Cytotoxic T-Lymphocyte Antigen 4 (CTLA-4); and (b) an ectonucleotide pyrophosphatase/phosphodiesterase family member 1 (ENPP1) inhibitor.

In an embodiment, the antibody is administered at a dose of about 25 mg to about 250 mg. In an embodiment, the antibody is administered at a dose of about 25 mg to about 150 mg. In an embodiment, the antibody is administered at a dose of about 50 mg to about 150 mg. In an embodiment, the antibody is administered at a dose of about 75 mg to about 150 mg.

In an embodiment, the antibody is administered at a dose of 25 mg to 250 mg. In an embodiment, the antibody is administered at a dose of 50 mg to 175 mg. In an embodiment, the antibody is administered at a dose of 25 mg to 150 mg. In an embodiment, the antibody is administered at a dose of 50 mg to 150 mg. In an embodiment, the antibody is administered at a dose of 75 mg to 150 mg.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg.

In an embodiment, the antibody is administered at a dose of about 25 mg to about 250 mg, once weekly. In an embodiment, the antibody is administered at a dose of about 25 mg to about 150 mg, once weekly. In an embodiment, the antibody is administered at a dose of about 50 mg to about 150 mg, once weekly. In an embodiment, the antibody is administered at a dose of about 75 mg to about 150 mg, once weekly.

In an embodiment, the antibody is administered at a dose of 25 mg to 250 mg, once weekly. In an embodiment, the antibody is administered at a dose of 50 mg to 175 mg, once weekly. In an embodiment, the antibody is administered at a dose of 25 mg to 150 mg, once weekly. In an embodiment, the antibody is administered at a dose of 50 mg to 150 mg, once weekly. In an embodiment, the antibody is administered at a dose of 75 mg to 150 mg, once weekly.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg, once weekly.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg, once weekly.

In an embodiment, the antibody is administered at a dose of about 25 mg to about 250 mg, once every 2 weeks. In an embodiment, the antibody is administered at a dose of about 25 mg to about 150 mg, once every 2 weeks. In an embodiment, the antibody is administered at a dose of about 50 mg to about 150 mg, once every 2 weeks. In an embodiment, the antibody is administered at a dose of about 75 mg to about 150 mg, once every 2 weeks.

In an embodiment, the antibody is administered at a dose of 25 mg to 250 mg, once every 2 weeks. In an embodiment, the antibody is administered at a dose of 50 mg to 175 mg, once every 2 weeks. In an embodiment, the antibody is administered at a dose of 25 mg to 150 mg, once every 2 weeks. In an embodiment, the antibody is administered at a dose of 50 mg to 150 mg, once every 2 weeks. In an embodiment, the antibody is administered at a dose of 75 mg to 150 mg, once every 2 weeks.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg, once every 2 weeks.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg, once every 2 weeks.

In an embodiment, the antibody is administered at a dose of about 25 mg to about 250 mg, once every 3 weeks. In an embodiment, the antibody is administered at a dose of about 25 mg to about 150 mg, once every 3 weeks. In an embodiment, the antibody is administered at a dose of about 50 mg to about 150 mg, once every 3 weeks. In an embodiment, the antibody is administered at a dose of about 75 mg to about 150 mg, once every 3 weeks.

In an embodiment, the antibody is administered at a dose of 25 mg to 250 mg, once every 3 weeks. In an embodiment, the antibody is administered at a dose of 50 mg to 175 mg, once every 3 weeks. In an embodiment, the antibody is administered at a dose of 25 mg to 150 mg, once every 3 weeks. In an embodiment, the antibody is administered at a dose of 50 mg to 150 mg, once every 3 weeks. In an embodiment, the antibody is administered at a dose of 75 mg to 150 mg, once every 3 weeks.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg, once every 3 weeks.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg, once every 3 weeks.

In an embodiment, the antibody is administered at a dose of about 25 mg to about 250 mg, once every 4 weeks. In an embodiment, the antibody is administered at a dose of about 25 mg to about 150 mg, once every 4 weeks. In an embodiment, the antibody is administered at a dose of about 50 mg to about 150 mg, once every 4 weeks. In an embodiment, the antibody is administered at a dose of about 75 mg to about 150 mg, once every 4 weeks.

In an embodiment, the antibody is administered at a dose of 25 mg to 250 mg, once every 4 weeks. In an embodiment, the antibody is administered at a dose of 50 mg to 175 mg, once every 4 weeks. In an embodiment, the antibody is administered at a dose of 25 mg to 150 mg, once every 4 weeks. In an embodiment, the antibody is administered at a dose of 50 mg to 150 mg, once every 4 weeks. In an embodiment, the antibody is administered at a dose of 75 mg to 150 mg, once every 4 weeks.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg, once every 4 weeks.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg, once every 4 weeks.

In an embodiment, the antibody is administered at a dose of about 25 mg to about 250 mg, once every 5 weeks. In an embodiment, the antibody is administered at a dose of about 25 mg to about 150 mg, once every 5 weeks. In an embodiment, the antibody is administered at a dose of about 50 mg to about 150 mg, once every 5 weeks. In an embodiment, the antibody is administered at a dose of about 75 mg to about 150 mg, once every 5 weeks.

In an embodiment, the antibody is administered at a dose of 25 mg to 250 mg, once every 5 weeks. In an embodiment, the antibody is administered at a dose of 50 mg to 175 mg, once every 5 weeks. In an embodiment, the antibody is administered at a dose of 25 mg to 150 mg, once every 5 weeks. In an embodiment, the antibody is administered at a dose of 50 mg to 150 mg, once every 5 weeks. In an embodiment, the antibody is administered at a dose of 75 mg to 150 mg, once every 5 weeks.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg, once every 5 weeks.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg, once every 5 weeks.

In an embodiment, the antibody is administered at a dose of about 25 mg to about 250 mg, once every 6 weeks. In an embodiment, the antibody is administered at a dose of about 25 mg to about 150 mg, once every 6 weeks. In an embodiment, the antibody is administered at a dose of about 50 mg to about 150 mg, once every 6 weeks. In an embodiment, the antibody is administered at a dose of about 75 mg to about 150 mg, once every 6 weeks.

In an embodiment, the antibody is administered at a dose of 25 mg to 250 mg, once every 6 weeks. In an embodiment, the antibody is administered at a dose of 50 mg to 175 mg, once every 6 weeks. In an embodiment, the antibody is administered at a dose of 25 mg to 150 mg, once every 6 weeks. In an embodiment, the antibody is administered at a dose of 50 mg to 150 mg, once every 6 weeks. In an embodiment, the antibody is administered at a dose of 75 mg to 150 mg, once every 6 weeks.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg, once every 6 weeks.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg, once every 6 weeks.

In an embodiment, the antibody is administered at a dose of about 25 mg to about 250 mg, once every 7 weeks. In an embodiment, the antibody is administered at a dose of about 25 mg to about 150 mg, once every 7 weeks. In an embodiment, the antibody is administered at a dose of about 50 mg to about 150 mg, once every 7 weeks. In an embodiment, the antibody is administered at a dose of about 75 mg to about 150 mg, once every 7 weeks.

In an embodiment, the antibody is administered at a dose of 25 mg to 250 mg, once every 7 weeks. In an embodiment, the antibody is administered at a dose of 50 mg to 175 mg, once every 7 weeks. In an embodiment, the antibody is administered at a dose of 25 mg to 150 mg, once every 7 weeks. In an embodiment, the antibody is administered at a dose of 50 mg to 150 mg, once every 7 weeks. In an embodiment, the antibody is administered at a dose of 75 mg to 150 mg, once every 7 weeks.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg, once every 7 weeks.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg, once every 7 weeks.

In an embodiment, the antibody is administered at a dose of about 25 mg to about 250 mg, once every 8 weeks. In an embodiment, the antibody is administered at a dose of about 25 mg to about 150 mg, once every 8 weeks. In an embodiment, the antibody is administered at a dose of about 50 mg to about 150 mg, once every 8 weeks. In an embodiment, the antibody is administered at a dose of about 75 mg to about 150 mg, once every 8 weeks.

In an embodiment, the antibody is administered at a dose of 25 mg to 250 mg, once every 8 weeks. In an embodiment, the antibody is administered at a dose of 50 mg to 175 mg, once every 8 weeks. In an embodiment, the antibody is administered at a dose of 25 mg to 150 mg, once every 8 weeks. In an embodiment, the antibody is administered at a dose of 50 mg to 150 mg, once every 8 weeks. In an embodiment, the antibody is administered at a dose of 75 mg to 150 mg, once every 8 weeks.

In an embodiment, the antibody is administered at a dose of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg, once every 8 weeks.

In an embodiment, the antibody is administered at a dose of 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg, once every 8 weeks.

In an embodiment, the antibody is administered intravenously. In an embodiment, the antibody is administered intratumorally.

In an embodiment, the antibody is administered by intravenous infusion over about 30 minutes. In an embodiment, the antibody is administered by intravenous infusion over about 45 minutes. In an embodiment, the antibody is administered by intravenous infusion over about 60 minutes. In an embodiment, the antibody is administered by intravenous infusion over about 90 minutes.

In an embodiment, the antibody is administered about once weekly. In an embodiment, the antibody is administered about once every 2 weeks. In an embodiment, the antibody is administered about once every 3 weeks. In an embodiment, the antibody is administered about once every 4 weeks. In an embodiment, the antibody is administered about once every 5 weeks. In an embodiment, the antibody is administered about once every 6 weeks. In an embodiment, the antibody is administered about once every 7 weeks. In an embodiment, the antibody is administered about once every 8 weeks.

In an embodiment, the antibody is administered once weekly. In an embodiment, the antibody is administered once every 2 weeks. In an embodiment, the antibody is administered once every 3 weeks. In an embodiment, the antibody is administered once every 4 weeks. In an embodiment, the antibody is administered once every 5 weeks. In an embodiment, the antibody is administered once every 6 weeks. In an embodiment, the antibody is administered once every 7 weeks. In an embodiment, the antibody is administered once every 8 weeks.

In an embodiment, the ENPP1 inhibitor is administered to the subject at a dose of about 5 mg/day to about 1000 mg/day. In an embodiment, the ENPP1 inhibitor is administered to the subject at a dose of about 100 mg/day to about 500 mg/day. In an embodiment, the ENPP1 inhibitor is administered to the subject at a dose of about 10 mg/day to about 100 mg/day. In an embodiment, the ENPP1 inhibitor is administered to the subject at a dose of about 10 mg/day to about 50 mg/day.

In an embodiment, the ENPP1 inhibitor is administered to the subject at a dose of 5 mg/day to 1000 mg/day. In an embodiment, the ENPP1 inhibitor is administered to the subject at a dose of 100 mg/day to 500 mg/day. In an embodiment, the ENPP1 inhibitor is administered to the subject at a dose of 10 mg/day to 100 mg/day. In an embodiment, the ENPP1 inhibitor is administered to the subject at a dose of 10 mg/day to 50 mg/day.

In an embodiment, the ENPP1 inhibitor is administered to the subject at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In an embodiment, the ENPP1 inhibitor is administered to the subject at a dose of 10 mg/day, 20 mg/day, 30 mg/day, 40 mg/day, 50 mg/day, 60 mg/day, 70 mg/day, 80 mg/day, 90 mg/day, 100 mg/day, 200 mg/day, 300 mg/day, 400 mg/day, 500 mg/day, 600 mg/day, 700 mg/day, 800 mg/day, 900 mg/day, or 1000 mg/day.

In an embodiment, the antibody and the ENPP1 inhibitor are administered to the subject at substantially the same time. In an embodiment, the antibody and the ENPP1 inhibitor are administered to the subject at different times.

In an embodiment, the antibody is administered to the subject before the ENPP1 inhibitor. In an embodiment, the ENPP1 inhibitor is administered to the subject before the antibody.

In an embodiment, the method results in about a 1, 5, 10, 20, 30, 40, 50, 60, 70, 80, 90, or 100% reduction in tumor burden in the subject. In an embodiment, the method results in no change in tumor burden in the subject. In an embodiment, the method results in about a 1% reduction in tumor burden in the subject. In an embodiment, the method results in about a 5% reduction in tumor burden in the subject. In an embodiment, the method results in about a 10% reduction in tumor burden in the subject. In an embodiment, the method results in about a 20% reduction in tumor burden in the subject. In an embodiment, the method results in about a 30% reduction in tumor burden in the subject. In an embodiment, the method results in about a 40% reduction in tumor burden in the subject. In an embodiment, the method results in about a 50% reduction in tumor burden in the subject. In an embodiment, the method results in about a 60% reduction in tumor burden in the subject. In an embodiment, the method results in about a 70% reduction in tumor burden in the subject. In an embodiment, the method results in about an 80% reduction in tumor burden in the subject. In an embodiment, the method results in about a 90% reduction in tumor burden in the subject. In an embodiment, the method results in about a 100% reduction in tumor burden in the subject.

In an embodiment, the method results in a reduced tumor burden. In an embodiment, the method results in increased survival. In an embodiment, the method results in an increase in overall survival. In an embodiment, the method results in an increase in progression-free survival.

In an embodiment, the cancer is bladder cancer, breast cancer, cervical cancer, colorectal cancer, colon adenocarcinoma, head and neck cancer, leukemia, lymphoma, tenosynovial giant cell tumor, gastric cancer, gastroesophageal cancer, glioblastoma, sarcoma, pancreatic cancer, melanoma, mesothelioma, renal cell adenocarcinoma, hepatocellular carcinoma, stomach adenocarcinoma, kidney renal clear cell carcinoma, esophageal carcinoma, ovarian cancer, small cell lung cancer, non-small cell lung cancer, or lung adenocarcinoma.

In an embodiment, the cancer is relapsed and/or refractory. In an embodiment, the cancer is metastatic. In an embodiment, the cancer is microsatellite stable colorectal cancer. In an embodiment, the cancer is renal cell carcinoma. In an embodiment, the cancer is hepatocellular carcinoma. In an embodiment, the cancer is pancreatic cancer.

In an embodiment, the subject has received at least one prior immunotherapy or chemotherapy. In an embodiment, the subject has not received any prior chemotherapy, radiotherapy, or immunotherapy. In an embodiment, the cancer is refractory to a standard of care treatment. In an embodiment, the standard of care treatment is chemotherapy, immunotherapy, or radiation.

In an embodiment, the method further comprises administering an additional therapeutic agent to the subject.

In an embodiment, the additional therapeutic agent is a checkpoint targeting agent selected from the group consisting of an antagonist anti-PD-1 antibody, an antagonist anti-PD-L1 antibody, an antagonist anti-PD-L2 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, an agonist anti-CD28 antibody. In an embodiment, the additional therapeutic agent is radiotherapy.

In an embodiment, the additional therapeutic agent is an antibody that specifically binds to human PD-1. In an embodiment, the additional therapeutic agent is nivolumab, pembrolizumab, dostarlimab, or balstilimab.

In an embodiment, the method reduces tumor size in the subject. In an embodiment, the method increases T-cell activation in the subject.

In an embodiment of each of the foregoing methods and uses, the subject is a human subject.

In an aspect, provided herein is a method of treating cancer in a subject in need thereof, the method comprising administering to the subject a therapeutically effective amount of: (a) an antibody that specifically binds to human Cytotoxic T-Lymphocyte Antigen 4 (CTLA-4), wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8; and (b) the ectonucleotide pyrophosphatase/phosphodiesterase family member 1 (ENPP1) inhibitor:

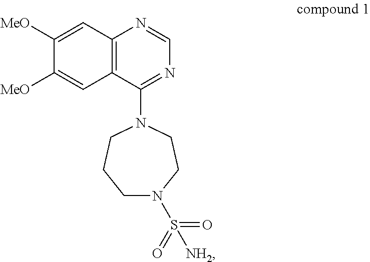 [SMILES: COc1cc2ncnc(N3CCCN(S(N)(=O)=O)CC3)c2cc1OC]

or a pharmaceutically acceptable salt thereof.

In an aspect, provided herein is a method of enhancing the activation of T cells in a subject who has cancer, the method comprising administering to the subject a therapeutically effective amount of: (a) an antibody that specifically binds to human Cytotoxic T-Lymphocyte Antigen 4 (CTLA-4), wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8; and (b) the ectonucleotide pyrophosphatase/phosphodiesterase family member 1 (ENPP1) inhibitor:

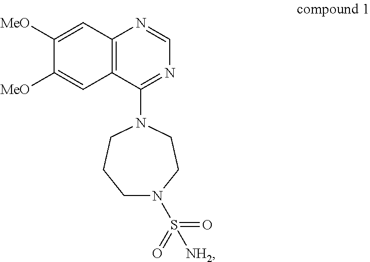 [SMILES: COc1cc2ncnc(N3CCCN(S(N)(=O)=O)CC3)c2cc1OC]

or a pharmaceutically acceptable salt thereof.

In an aspect, disclosed herein is an antibody that specifically binds to human CTLA-4 and an ENPP1 inhibitor for use in the treatment of cancer, wherein the treatment is performed according to any one of the methods described herein.

In an aspect, disclosed herein is an antibody that specifically binds to human CTLA-4 and an ENPP1 inhibitor for use in the manufacture of a medicament for the treatment of cancer, wherein the treatment is performed according to any one of the methods described herein.

In an aspect, disclosed herein is a use of an antibody that specifically binds to human CTLA-4 and an ENPP1 inhibitor for the treatment of cancer, wherein the treatment is performed according to any one of the methods described herein.

In an aspect, disclosed herein is an antibody that specifically binds to human CTLA-4 and an ENPP1 inhibitor for use in enhancing the activation of T cells, wherein the treatment is performed according to any one of the methods described herein.

In an aspect, disclosed herein is an antibody that specifically binds to human CTLA-4 and an ENPP1 inhibitor for use in the manufacture of a medicament for enhancing the activation of T cells, wherein the treatment is performed according to any one of the methods described herein.

In an aspect, disclosed herein is a use of an antibody that specifically binds to human CTLA-4 and an ENPP1 inhibitor for enhancing the activation of T cells, wherein the treatment is performed according to any one of the methods described herein.

### Therapeutic Combinations

In an aspect, disclosed herein is a therapeutic combination comprising: (a) an antibody that specifically binds to human Cytotoxic T-Lymphocyte Antigen 4 (CTLA-4); and (b) an ectonucleotide pyrophosphatase/phosphodiesterase family member 1 (ENPP1) inhibitor.

In an aspect, disclosed herein is a therapeutic combination comprising: (a) 25 mg to 250 mg of an antibody that specifically binds to human CTLA-4; and (b) an ENPP1 inhibitor.

In an aspect, disclosed herein is a therapeutic combination comprising: (a) about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg of an antibody that specifically binds to human CTLA-4; and (b) an ENPP1 inhibitor.

In an aspect, disclosed herein is a therapeutic combination comprising: (a) 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg of an antibody that specifically binds to human CTLA-4; and (b) an ENPP1 inhibitor.

In an aspect, disclosed herein is a therapeutic combination comprising: (a) 25 mg to 250 mg of an antibody that specifically binds to human CTLA-4; and (b) 5 mg to 1000 mg of an ENPP1 inhibitor.

In an aspect, disclosed herein is a therapeutic combination comprising: (a) about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 175 mg, about 200 mg, about 225 mg, or about 250 mg of an antibody that specifically binds to human CTLA-4; and (b) 5 mg to 1000 mg of an ENPP1 inhibitor.

In an aspect, disclosed herein is a therapeutic combination comprising: (a) 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 175 mg, 200 mg, 225 mg, or 250 mg of an antibody that specifically binds to human CTLA-4; and (b) 5 mg to 1000 mg of an ENPP1 inhibitor.

In an embodiment, the antibody comprises a human IgG₁ heavy chain constant region that is a variant of a wild-type human IgG heavy chain constant region, wherein the variant binds to FcγRIIIA with a higher affinity than the wild-type human IgG₁ heavy chain constant region binds to FcγRIIIA.

In an embodiment, the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a human IgG1 heavy chain constant region comprising S239D/A330L/I332E mutations, numbered according to the EU numbering system. In an embodiment, the antibody comprises a heavy chain comprising the amino acid sequence set forth in SEQ ID NO: 9 and a light chain comprising the amino acid sequence set forth in SEQ ID NO: 10.

In an embodiment, the antibody is botensilimab.

In an embodiment, the ENPP1 inhibitor is:

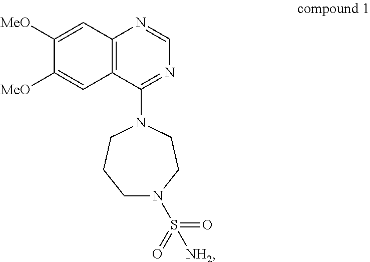 [SMILES: COc1cc2ncnc(N3CCCN(S(N)(=O)=O)CC3)c2cc1OC]

or a pharmaceutically acceptable salt thereof.

In an embodiment, the therapeutic combination further comprises a pharmaceutically acceptable carrier or excipient.

In an embodiment, the combination is for use in treating cancer. In an embodiment, the combination is for use in enhancing the activation of T cells in a subject who has cancer.

In an embodiment, the antibody and the ENPP1 inhibitor are formulated into a single composition. In an embodiment, the antibody and the ENPP1 inhibitor are formulated in separate compositions.

### EXAMPLES

### Example 1—Botensilimab (AGEN1181) in Combination with an ENPP1 Inhibitor Suppresses Tumor Growth and Increases Immune Infiltration in a Mouse Model of Cancer

The combination of an anti-CTLA-4 antibody and an ENPP1 inhibitor was analyzed for its efficacy in inhibiting tumor growth in a mouse model of cancer.

### Tumor Growth

BALB/cJ mice were subcutaneously implanted with CT-26 cells (mouse colorectal cancer cells; 2.5×10⁵ per mouse) along with matrigel (Corning #: 354230) (1:1 ratio) into the right hind flank. Once tumors were established on day 8, the mice were administered 0.2 mg/kg of compound 1:

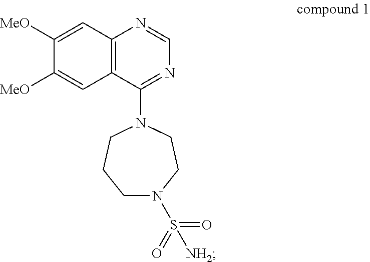 [SMILES: COc1cc2ncnc(N3CCCN(S(N)(=O)=O)CC3)c2cc1OC]

(or 1% methylcellulose alone for the vehicle control) orally, twice per day, which was continued until day 30. On day 12, mice were given a single IP dose of a mouse surrogate of the anti-CTLA-4 antibody botensilimab (botensilimab^{ms}) or In Vivo Mab mouse IgG2b isotype control (Bio X Cell, Cat #: BE0086) in Dilution Buffer (Bio X Cell, Cat #: IP0070). Body weights and tumor diameter were measured three times weekly for the duration of the study. Tumor volume was determined by calculating the volume of an ellipsoid using the formula: length×width²×0.5. Data are expressed as mean±SEM from n=15 mice/group;*p<0.05, **p<0.01, ****p<0.0001; one-way ANOVA, Tukey's multiple comparisons test.

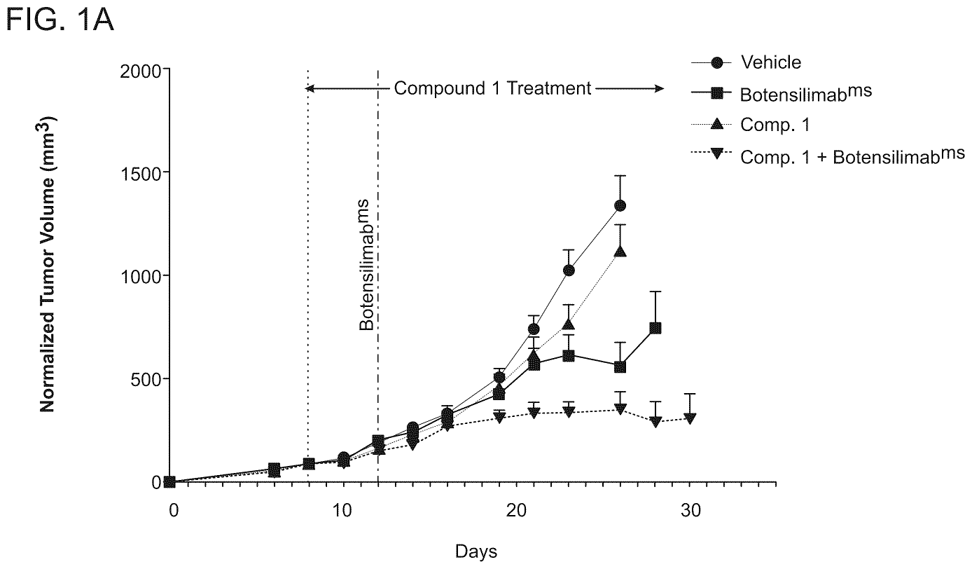

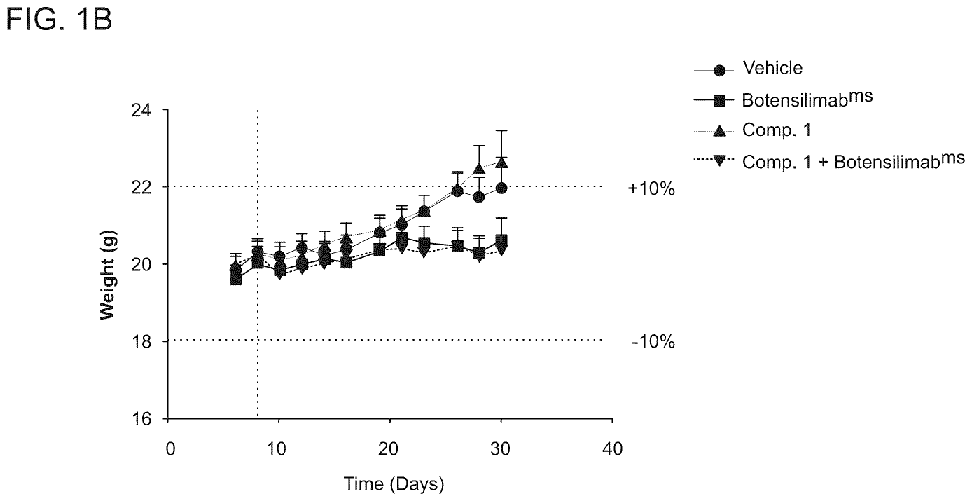

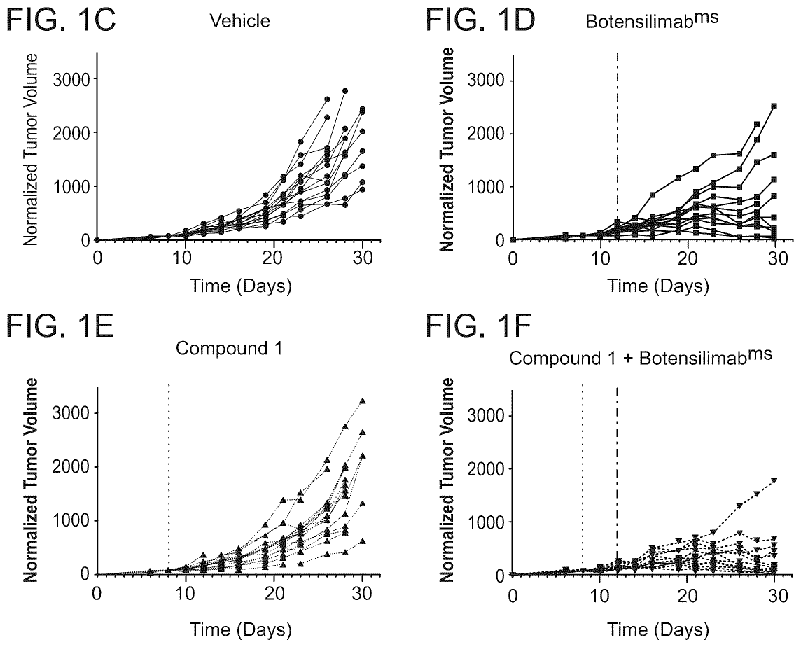

The results in FIG. 1A and FIGS. 1C-1F show that the combination of compound 1 and botensilimab^{ms} (see, FIGS. 1A and 1F) suppressed tumor growth to a greater extent than either compound 1 monotherapy (see, FIGS. 1A and 1E) or botensilimab^{ms} monotherapy (see, FIGS. 1A and 1D). All conditions suppressed tumor growth more than vehicle controls (FIG. 1C). FIG. 1B shows that the mice had normal body weights in all treatment groups, which indicates that none of the treatments were significantly toxic to the mice.

### Tumor Tissue Processing and Immunohistochemistry

The tumors were collected at day 30 and analyzed for CD3+ and CD8+ T-cell infiltration by IHC to determine the T cell activation in mice following treatment with compound 1 alone, botensilimab^{ms} alone, or the combination of compound 1 and botensilimab^{ms}.

Tumors were fixed in a 10% neutral buffered formalin solution (Fisher Scientific #22-126-347), processed and embedded in paraffin blocks and sectioned at 4 micron. The sections were deparaffinized with xylenes, rehydrated through graded ethanol using a BondMax autostainer (Leica Biosystems), antigen retrieval with Rodent Decloaker (Biocare #RD913) using a decloaking chamber (Biocare #DC2002) and blocked with Rodent Block M (Biocare #RBM961) and Peroxidazed 1 (Biocare #RD913). Immunohistochemical staining was performed with primary antibodies CD3 (Abcam, #ab16669) and CD8 (Cell signaling #98941) incubation for 1 hour, Rabbit HRP (Cell Signaling #8114S) for 30 minutes and developed with DAB Substrate Kit (Cell Signaling #8059S) followed by CAT Hematoxylin (Biocare, #CATHAE). Slides were scanned with Aperio Scanscope (Leica Biosystems) and analyzed using QuPath by quantifying at least 3 images from each sample. Data are expressed as mean+SD from n=6 mice/group; *p<0.05, **p<0.01, ****p<0.0001; one-way ANOVA, Tukey's multiple comparisons test.

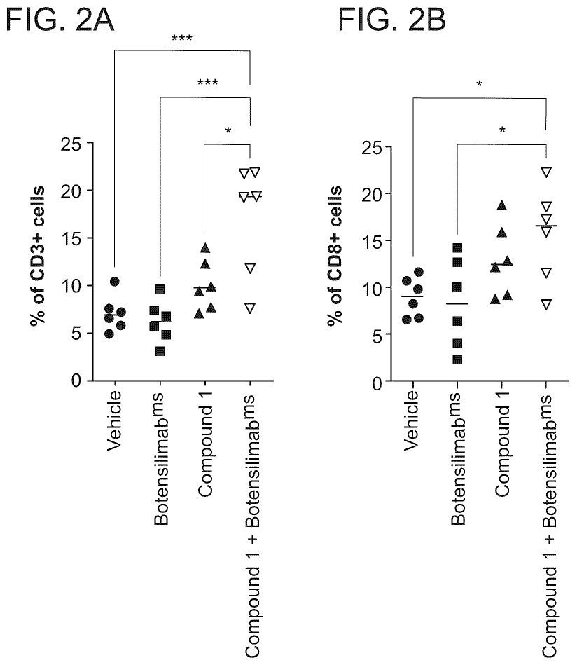

FIGS. 2A and 2B show that the mice treated with a combination of compound 1 and botensilimab^{ms} exhibited significantly increased CD3+ (FIG. 2A) and CD8+ T cell (FIG. 2B) infiltration in tumors compared to botensilimab^{ms} alone.

RNA Extraction, cDNA Synthesis, and rtPCR

The tumor samples from day 30 were also analyzed for the expression of markers of immune activation, including IFNβ, IFNγ, CXCL10, ISG56, and Granzyme B.

RNA extractions from tumor samples were performed according to the RNeasy Mini Kit (Qiagen) manufacturer's instructions, cDNA synthesis was performed according to the High Capacity cDNA Reverse transcription kit (ThermoFisher) manufacturer's instructions, and quantification was determined using a Nanodrop 8000. Real time PCR (RT-PCR) was performed in quadruplicate using 400 ng of cDNA per reaction and SYBR Green PCR Master Mix (ThermoFisher) on the Applied Biosystems Viia7. Samples were normalized to GAPDH gene expression to determine ΔCT, to vehicle controls to determine ΔΔCT, and presented as fold change gene expression. Data are expressed as mean±SD from n=6 mice/group; *p<0.05, **p<0.01, ****p<0.0001; one-way ANOVA, Tukey's multiple comparisons test.

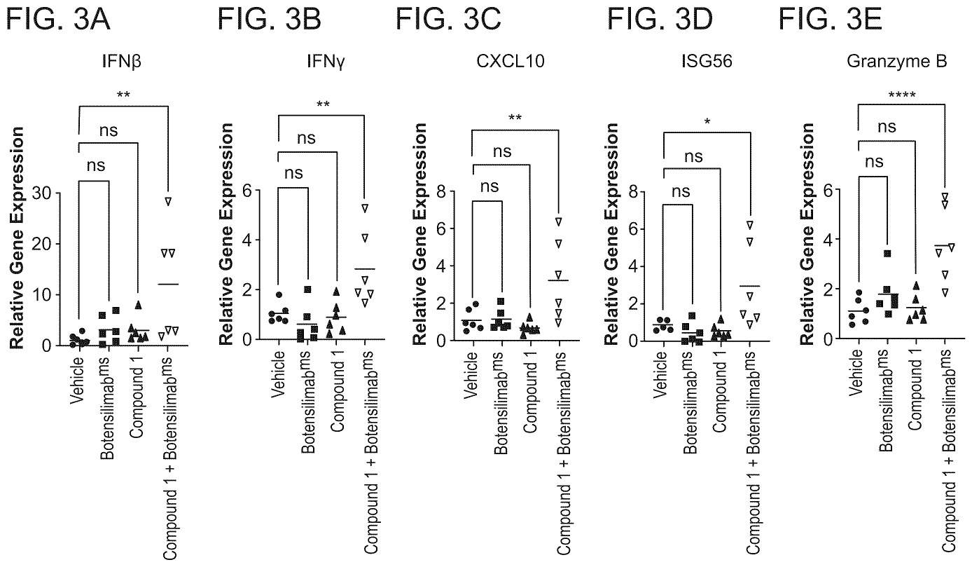

FIGS. 3A-3E show significantly increased expression of all of the immune activation genes analyzed in the mice treated with the combination of compound 1 and botensilimab^{ms}. Specifically, the expression of IFNβ (FIG. 3A), IFNγ (FIG. 3B), CXCL10 (FIG. 3C), ISG56 (FIG. 3D), and Granzyme B (FIG. 3E) were significantly increased in the mice treated with the combination of compound 1 and botensilimab^{ms}, compared to either monotherapy alone, or vehicle controls.

The above observations were bolstered by further studies directed to the early assessment of compound 1 and botensilimab^{ms} treatment on effecting changes in innate immune responses.

For these studies, similar to the above, female BALB/cJ mice, subcutaneously implanted with CT-26 cells into the right hind flank, were treated with 0.2 mg/kg of compound 1 orally, twice daily for 8 days, and/or a single dose of botensilimab^{ms} (100 μg) was administered at day 3 after the initiation of dosing. Tumor samples were processed and analyzed using the same methods as described above.

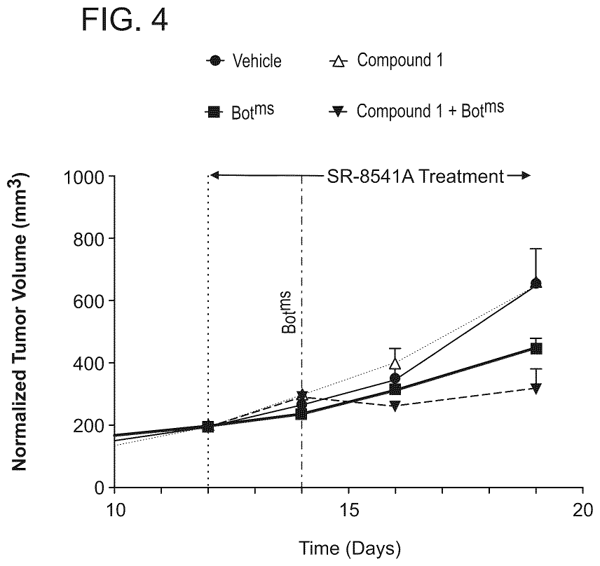

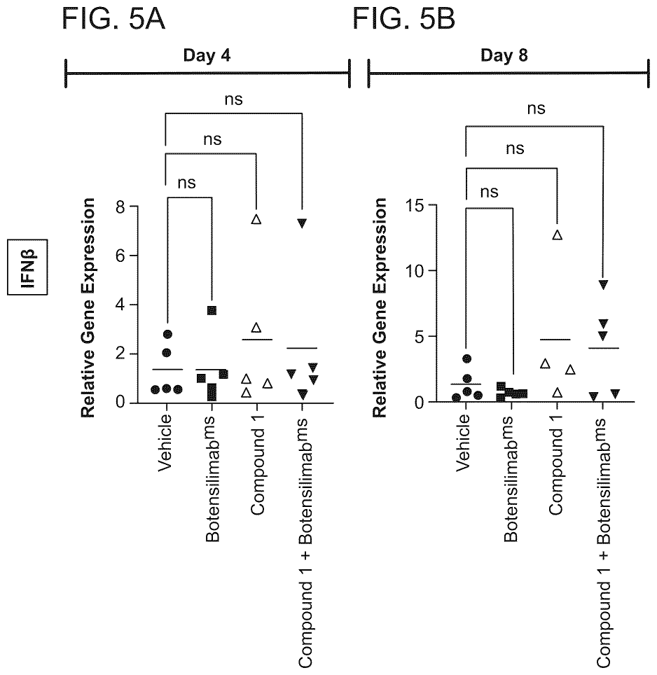

FIG. 4 shows that the combination of compound 1 and botensilimab^{ms} had an effect on tumor growth inhibition to a greater extent than either treatment with compound 1 alone, or botensilimab^{ms} alone.

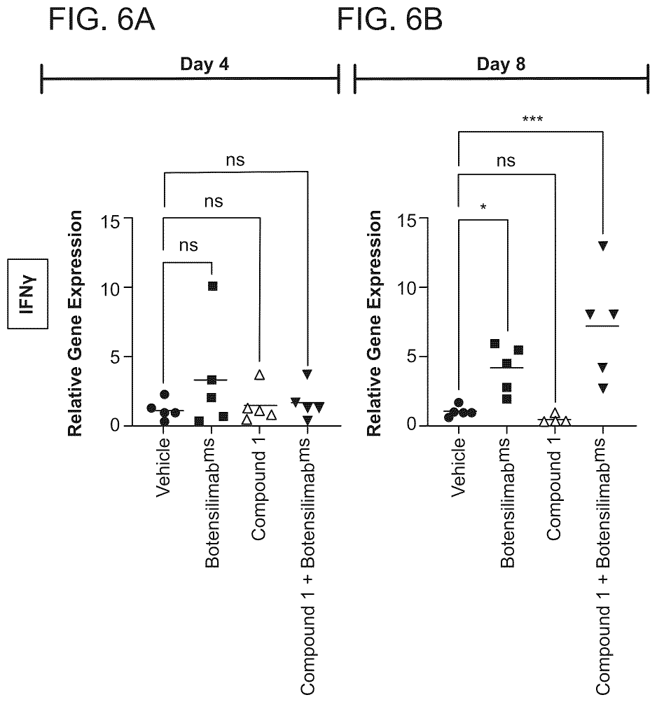

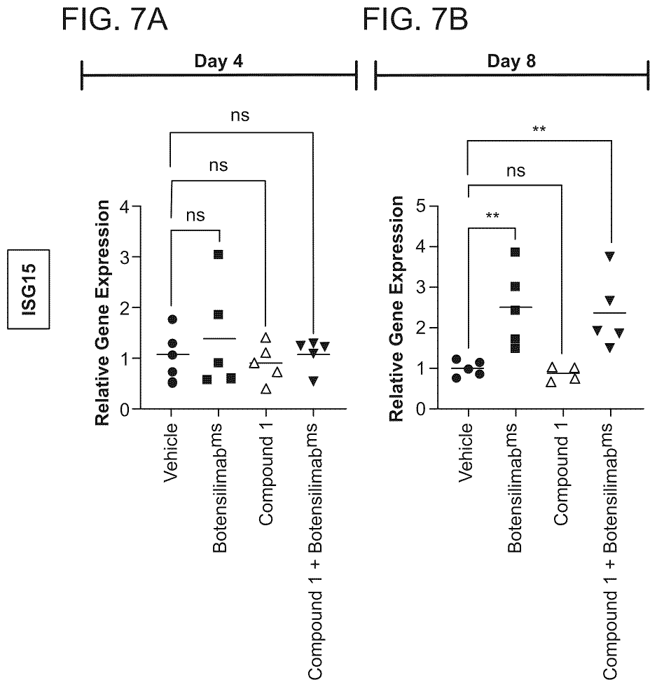

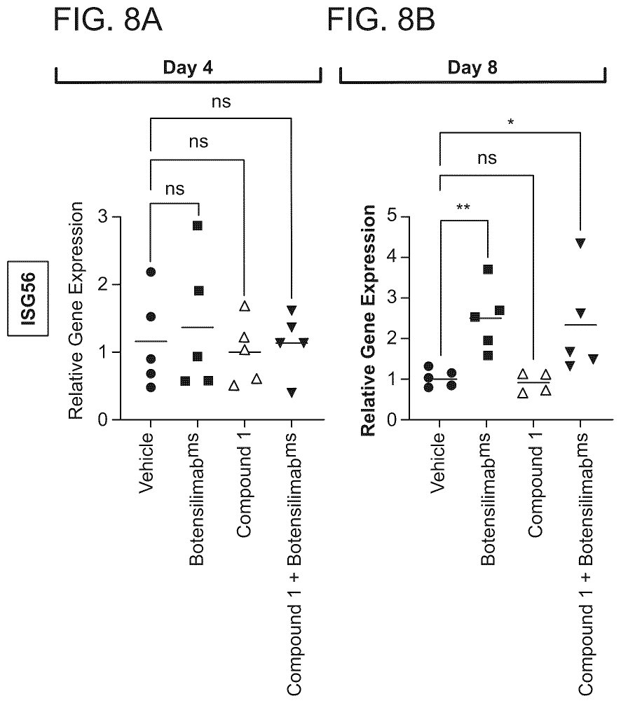

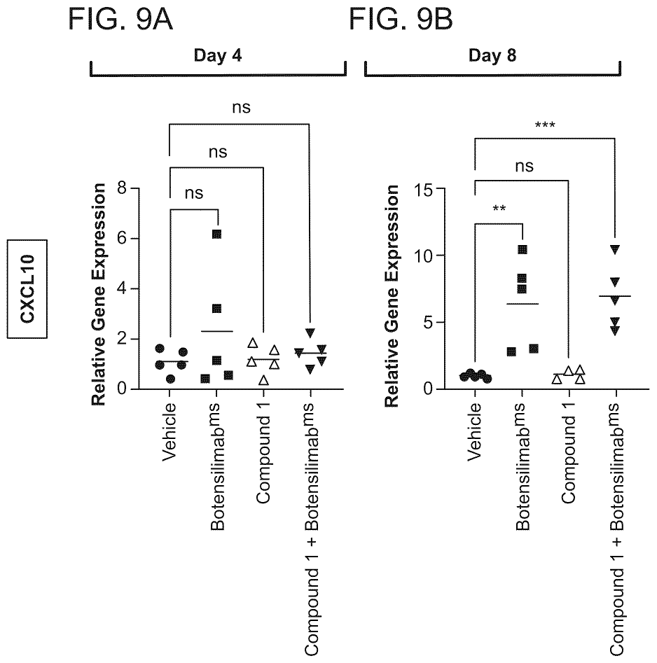

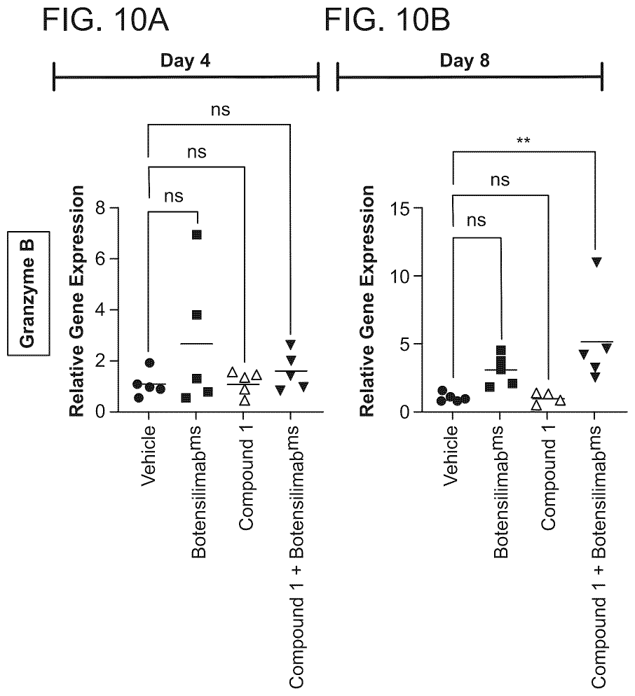

Tumor samples from day 4 and day 8 after initiation of dosing were analyzed for the expression of immune pathway markers IFNβ, IFNγ, ISG15, ISG56, CXCL10, and Granzyme B, using RT-PCR, as described above. Significantly increased expression of IFNγ (FIG. 6B), ISG15 (FIG. 7B), ISG56 (FIG. 8B), CXCL10 (FIG. 9B), and Granzyme B (FIG. 10B) was observed on day 8 after initiation of dosing in mice treated with the combination of compound 1 and botensilimab^{ms}, over expression of the markers on day 4 (FIGS. 6A, 7A, 8A, 9A, and 10A, respectively), indicating the generation of an early, robust immune response to the combination treatment.

### Conclusion

The combination of an ENPP1 inhibitor and an anti-CTLA-4 antibody exhibited a significant increase in overall efficacy, along with increased levels of CD3+ and CD8+ T-cell infiltration into the tumors, and increased levels of markers of immune activation in a mouse model of colon cancer. These results indicate that stimulating both the innate and adaptive immune responses is a promising approach for treating cancer.

### Example 2—an ENPP1 Inhibitor Enhances the Effects of Checkpoint Inhibitors in Human Cancer Cells

The combination of compound 1 (the ENPP1 inhibitor described in Example 1), an anti-PD-1 antibody, and an anti-CTLA-4 antibody is assessed for its effects on immune cell infiltration in human breast and ovarian cancer derived organoids.

Organoids derived from human breast cancer cells (MDA-MB-468 and MDA-MB-231) and ovarian cancer cells (SKOV3) are treated with compound 1, an anti-PD-1 antibody, an anti-CTLA-4 antibody (e.g., botensilimab), or a combination of compound 1, an anti-PD-1 antibody, and an anti-CTLA-4 antibody (e.g., botensilimab). The organoids are then incubated with RFP-labeled PBMCs for 48 hours. The RFP signal in the organoids is quantified using methods known in the art. It is expected that the organoids treated with the combination of compound 1, an anti-PD-1 antibody, and an anti-CTLA-4 antibody (e.g., botensilimab) have increased immune cell infiltration compared to the organoids treated with each monotherapy.

### Example 3—an ENPP1 Inhibitor Enhances the Effects of Botensilimab in Organoids Derived from Human Cancer Cells

The combination of compound 1 (the ENPP1 inhibitor described in Example 1) and botensilimab is assessed for its effects on immune cell infiltration in human breast and ovarian cancer derived organoids.

Organoids derived from human breast cancer cells (MDA-MB-468 and MDA-MB-231) and ovarian cancer cells (SKOV3) are treated with compound 1, botensilimab, or a combination of compound 1 and botonsilimab, and are incubated with RFP-labeled PBMCs for 48 hours. The RFP signal in the organoids is quantified using methods known in the art. It is expected that the organoids treated with the combination of compound 1 and botensilimab have increased immune cell infiltration compared to the organoids treated with each monotherapy.

### Example 4—Botensilimab (AGEN1181) in Combination with an ENPP1 Inhibitor and/or an Anti-PD-1 Antibody Suppresses Tumor Growth in a Syngeneic Mouse Model (CT-26) of Colorectal Cancer

The combination of an anti-CTLA-4 antibody, an ENPP1 inhibitor, and/or an anti-PD-1 antibody was analyzed for its efficacy in inhibiting tumor growth in a mouse model of cancer.

The same methods were used as described in Example 1. Briefly, female BALB/cJ mice were implanted subcutaneously with CT-26 cells into the right hind flank and treated with 0.2 mg/kg of compound 1 and/or 100 μg of botensilimab^{ms} and/or a monoclonal anti-PD-1 antibody (InVivoPlus anti-mouse PD-1 (CD279) antibody, clone RMP1-14, BioXCell, catalog #BP0146). Tumor growth and body weight were monitored over 40 days.

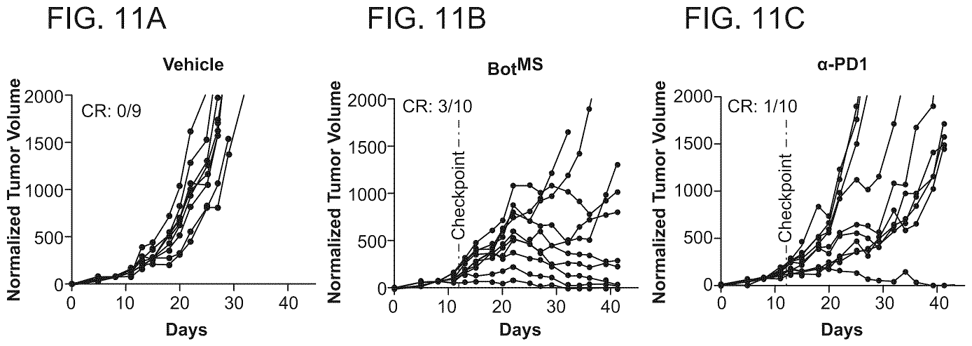

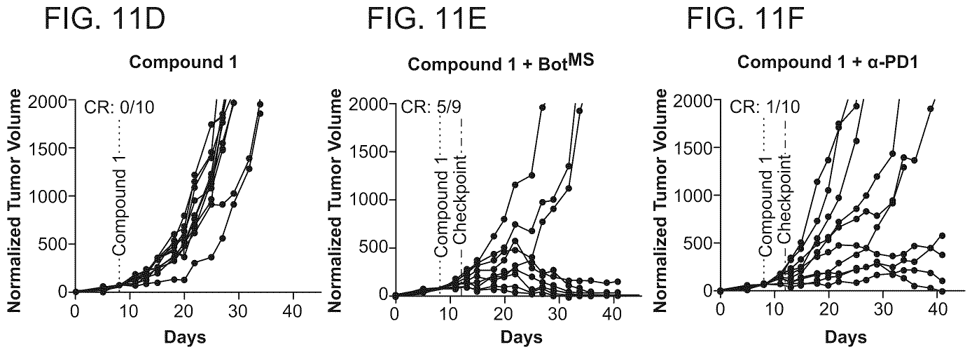

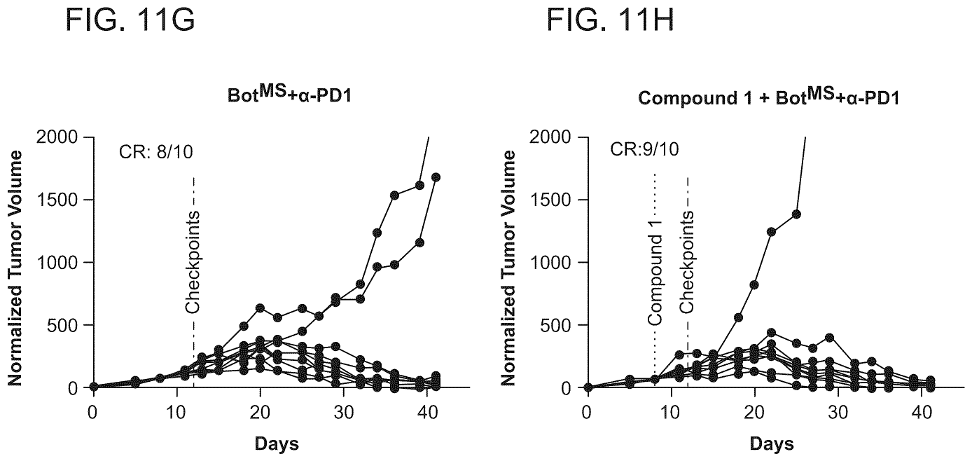

The synergistic effect between botensilimab^{ms} and compound 1 in the suppression of tumor growth described in Example 1 (see, e.g., FIGS. 1A and 1F) was confirmed (FIGS. 11B, 11D, and 11E). It was also demonstrated that the combination of botensilimab^{ms} and anti-PD-1 antibody resulted in greater suppression of tumor growth than with each monotherapy alone (FIGS. 11B, 11C, and 11G). The triple combination of botensilimab^{ms}, compound 1, and anti-PD-1 antibody, was found to further suppress tumor growth compared to each pairwise combination of botensilimab^{ms}, compound 1, and anti-PD-1 antibody (FIGS. 11E-11H). All conditions tested suppressed tumor growth more than vehicle controls (FIG. 11A). Treatment with the combination of compound 1 and botensilimab^{ms}, and with the triple combination of compound 1, botensilimab^{ms}, and anti-PD-1 antibody, resulted in the lowest change in body weight over time compared to treatment with either compound 1, botensilimab^{ms}, or anti-PD-1 alone, or pairwise combinations of compound 1, botensilimab^{ms}, and anti-PD-1 antibody.

### INCORPORATION BY REFERENCE

All patent and non-patent literature references cited above are incorporated herein by reference in their entirety.

1. A method of treating cancer in a subject in need thereof, the method comprising administering to the subject a therapeutically effective amount of:
(a) an antibody that specifically binds to human Cytotoxic T-Lymphocyte Antigen 4 (CTLA-4); and
(b) an ectonucleotide pyrophosphatase/phosphodiesterase family member 1 (ENPP1) inhibitor.

2. A method of enhancing the activation of T cells in a subject who has cancer, the method comprising administering to the subject a therapeutically effective amount of:
(a) an antibody that specifically binds to human Cytotoxic T-Lymphocyte Antigen 4 (CTLA-4); and
(b) an ectonucleotide pyrophosphatase/phosphodiesterase family member 1 (ENPP1) inhibitor.

3. The method of claim 1, wherein the antibody comprises a human IgG₁ heavy chain constant region that is a variant of a wild-type human IgG heavy chain constant region, wherein the variant binds to FcγRIIIA with a higher affinity than the wild-type human IgG₁ heavy chain constant region binds to FcγRIIIA.

4. The method of claim 1, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

5. The method of claim 1, wherein the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

6. The method of claim 1, wherein the antibody comprises a VH comprising the amino acid sequence set forth in SEQ ID NO: 7 and a VL comprising the amino acid sequence set forth in SEQ ID NO: 8.

7. The method of claim 1, wherein the antibody comprises a human IgG1 heavy chain constant region comprising S239D/A330L/I332E mutations, numbered according to the EU numbering system.

8. The method of claim 1, wherein the antibody comprises a heavy chain comprising the amino acid sequence set forth in SEQ ID NO: 9 and a light chain comprising the amino acid sequence set forth in SEQ ID NO: 10.

9. The method of claim 1, wherein the antibody that specifically binds to human CTLA-4 is botensilimab.

10. The method of claim 1, wherein the ENPP1 inhibitor is 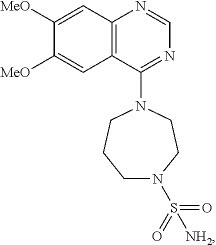 [SMILES: COc1cc2ncnc(N3CCCN(S(N)(=O)=O)CC3)c2cc1OC]
or a pharmaceutically acceptable salt thereof.

11. The method of claim 1, wherein the antibody is administered to the subject at a dose of 25 mg to 250 mg, optionally wherein the antibody is administered to the subject at a dose of 25 mg, 50 mg, 75 mg, 100 mg, or 150 mg.

12. (canceled)

13. The method of claim 1, wherein the antibody is administered to the subject intravenously, subcutaneously, or intratumorally.

14. The method of claim 1, wherein the antibody is administered to the subject once weekly, once every 2 weeks, once every 3 weeks, once every 4 weeks, once every 5 weeks, or once every 6 weeks.

15-19. (canceled)

20. The method of claim 1, wherein the ENPP1 inhibitor is administered to the subject at a dose of 5 mg/day to 1000 mg/day, optionally wherein the ENPP1 inhibitor is administered to the subject at a dose of 10 mg/day to 50 mg/day, further optionally wherein the ENPP1 inhibitor is administered to the subject at a dose of 10 mg/day, 20 mg/day, 30 mg/day, 40 mg/day, or 50 mg/day.

21. (canceled)

22. (canceled)

23. The method of claim 1, wherein the cancer
(a) is bladder cancer, breast cancer, cervical cancer, colorectal cancer, colon adenocarcinoma, head and neck cancer, leukemia, lymphoma, tenosynovial giant cell tumor, gastric cancer, gastroesophageal cancer, glioblastoma, sarcoma, pancreatic cancer, melanoma, mesothelioma, renal cell adenocarcinoma, hepatocellular carcinoma, stomach adenocarcinoma, kidney renal clear cell carcinoma, esophageal carcinoma, ovarian cancer, small cell lung cancer, non-small cell lung cancer, or lung adenocarcinoma;
(b) is relapsed and/or refractory;
(c) is metastatic; and/or
(d) is microsatellite stable colorectal cancer, renal cell carcinoma, hepatocellular carcinoma, or pancreatic cancer.

24-29. (canceled)

30. The method of claim 1, wherein
(a) the subject has received at least one prior immunotherapy or chemotherapy, optionally wherein the cancer is refractory to a standard of care treatment, further optionally wherein the standard of care treatment is chemotherapy, immunotherapy, or radiation; or
(b) the subject has not received any prior chemotherapy, radiotherapy, or immunotherapy.

31-33. (canceled)

34. The method of claim 1, wherein the method further comprises administering an additional therapeutic agent to the subject, optionally wherein the additional therapeutic agent is an antibody that specifically binds to human PD-1, further optionally wherein the additional therapeutic agent is nivolumab, pembrolizumab, dostarlimab, or balstilimab.

35. (canceled)

36. (canceled)

37. The method of claim 1, wherein the method reduces tumor size in the subject and/or increases T-cell activation in the subject.

38-41. (canceled)

42. A therapeutic combination comprising:
(a) an antibody that specifically binds to human Cytotoxic T-Lymphocyte Antigen 4 (CTLA-4); and
(b) an ectonucleotide pyrophosphatase/phosphodiesterase family member 1 (ENPP1) inhibitor.

43. The therapeutic combination of claim 42, wherein:
(a) the antibody comprises a human IgG₁ heavy chain constant region that is a variant of a wild-type human IgG heavy chain constant region, wherein the variant binds to FcγRIIIA with a higher affinity than the wild-type human IgG₁ heavy chain constant region binds to FcγRIIIA;
(b) the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8;
(c) the antibody comprises the CDRH1, CDRH2, CDRH3, CDRL1, CDRL2, and CDRL3 amino acid sequences set forth in SEQ ID NOs: 1, 2, 3, 4, 5, and 6, respectively;
(d) the antibody comprises a VH comprising the amino acid sequence set forth in SEQ ID NO: 7 and a VL comprising the amino acid sequence set forth in SEQ ID NO: 8;
(e) the antibody comprises a human IgG1 heavy chain constant region comprising S239D/A330L/I332E mutations, numbered according to the EU numbering system;
(f) the antibody comprises a heavy chain comprising the amino acid sequence set forth in SEQ ID NO: 9 and a light chain comprising the amino acid sequence set forth in SEQ ID NO: 10;
(g) the antibody is botensilimab; and/or
(h) the ENPP1 inhibitor is 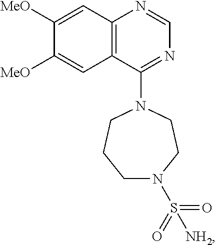 [SMILES: COc1cc2ncnc(N3CCCN(S(N)(=O)=O)CC3)c2cc1OC]
or a pharmaceutically acceptable salt thereof.

44-50. (canceled)


SEQUENCE LISTING (10 sequences)

SEQ ID NO: 1 - protein, 5 aa, synthetic construct
SYSMN

SEQ ID NO: 2 - protein, 17 aa, synthetic construct
SISSSSSYIYYAESVKG

SEQ ID NO: 3 - protein, 9 aa, synthetic construct
VGLFGPFDI

SEQ ID NO: 4 - protein, 11 aa, synthetic construct
RASQSVSRYLG

SEQ ID NO: 5 - protein, 7 aa, synthetic construct
GASTRAT

SEQ ID NO: 6 - protein, 9 aa, synthetic construct
QQYGSSPWT

SEQ ID NO: 7 - protein, 118 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSS

SEQ ID NO: 8 - protein, 107 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIK

SEQ ID NO: 9 - protein, 447 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC